SEQUENCE LISTING (1 sequence)

SEQ ID NO: 1 - DNA, 7587 bp, synthetic construct
  source 1..7587: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctgtacatttatattggctaatgtccaatatgaccgctatgttgacattgattattgactagttattaatagtaatcaattacggggtcattagttcatagcccatatatggacttccgcgttacataacttacggtaaatagcccgcctcgtgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccggccccctattgacgtcaatgacggtaaatggcccgcctggtattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaattgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatagctctttgccacaactatctctattggcttataccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgtctcggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgccgcgccaccagacataatagctgacagactaacagactgttcctttctatgggtcttttctgcagtcaccgtccttgacacgatagcttggtacagagctcggatctccattcgccattcaggctgcgcaactgttgggaaggggatcgtctagaggtacactcccaggatccaacttaaaaaaaaaaatcaggatccatccggagtaccggtctcagcttactacgcgcgcgctagcgtggtgttaatttgcagatatccagcactcgagtctagagggccctaggcaattaattaacggcggccgctctaaggtaaatataaaatttttaagtgtataatgtgttaaactactgattctaattgtttgtgtattttagatcacagtcccaaggctcatttcaggcccctcagtcctcacagtctgttcatgatcataatcagccataccacatttgtagaggttttacttgctttaaaaaacctcccacacctccccctgaacctgaaacataaaatgaatacaattgttgttgttaacttgtttattgcagcttataatagttacaaataaagcaatagcatcacaaatttcacaaataaagcatttttttcactgcattctagttgtggtttgtccaaactcatcaatatatcttatcagtttaaaccttcccacgtggcggagggactggggacccgggcacccgtcctgccccttcaccttccagctccgcctcctccgcgcggaccccgccccgtcccgacccctcccgggtccccggcccagccccctccaggccctcccagcccctccccttcctttccgcggccccgccctctcctcgcggcgcgagttttggaaagtccccaggctccccagcaggcagaagtatgcaaacgatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggactaggcttttgcaaaaagctcgtttaaactgtctgggccacagcttggcactgggatctgcgaacgcagcaagacgtagcccagcgcgtcggccccgagatgcgccgcgtgccgtgctggagatagcggaccgggtaggggaggcgcttttcccaaggcagtctggagcatgcgctttagcagccccgctgggcacttggcgctacacaagtggcctctggcctcgcacacattccatatccacgcgtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatagaagcgggtaggcctttggggcagcggccaatagcagctttgctccttcgctttctgggctcagaggctgggaaggggtgggtccgggggcgggctcaggggcgggctcaggggcggggcgggcgcccgaaggtcctcgcgaggcccggcattctgcacgcttcaaaagcgcacgtctgccgcgctgttctcctcttcctcatctccgggcctttcgtctagaatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctctattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaatctagattgtttattgcagcttataatggttacaaataaagcaatagcatcacaaatttcacaaataaagcatttttttcactgcattctagttgtggtttgtccaaactcatcaatgtatcttatatatagtcgacgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatcatccatgaccgagtacaagcccacggtgcgcctcgccacccgcgacgacgtccccagggccgtacgcaccctcgccgccgcgttcgccgactaccccgccacgcgccacaccgtcgatcctgaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgatgtcgaggtgcccgaaggaccgcgcacctggtgcatgacccgcaagcccggtgcctgaaacacggaaggagacaataccggaaggaaccgcgctatgacggcaataaaaagacagaataaaacgcacgggtgttgggtcgtttgttcagggtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2025/0146014 A1
(43) Pub. Date: May 8, 2025

(54) PLASMID FOR PRODUCTION OF RECOMBINANT PROTEIN IN MAMMALIAN CELL

(71) Applicant: IZMIR BIYOTIP VE GENOM MERKEZI, Izmir (TR)

(72) Inventors: Mehmet OZTURK, Izmir (TR); Aybike ERDOGAN, Izmir (TR)

(73) Assignee: IZMIR BIYOTIP VE GENOM MERKEZI, Izmir (TR)

(21) Appl. No.: 18/867,841

(22) PCT Filed: May 30, 2023
(86) PCT No.: PCT/TR2023/050484

§ 371 Date: Nov. 21, 2024

(30) Foreign Application Priority Data
Jun. 3, 2022 (TR) 2022/009209

(51) Int. Cl. C12N 15/85

(52) U.S. Cl. CPC C12N 15/85; C12N 2800/107; C12N 2830/15; C12N 2830/50; C12N 2830/60

(57) ABSTRACT

A plasmid developed for the production of recombinant protein in mammalian cells is provided. The plasmid vector includes the nucleic acid sequence with at least 80% similarity to SEQ ID NO: 1. The plasmid vector includes a CMV promoter, a CMV enhancer, CMV intron A, SV40 small t antigen intron, SV40 poly(A) signal, Syn21, and an hTERT-SV40 enhancer.

### CROSS REFERENCE TO THE RELATED APPLICATIONS

This application is the national phase entry of International Application No. PCT/TR2023/050484, filed on May 30, 2023, which is based upon and claims priority to Turkish Patent Application No. 2022/009209, filed on Jun. 3, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBUY182_Sequence_Listing.xml, created on Nov. 7, 2024, and is 9,308 bytes in size.

### TECHNICAL FIELD

The present invention relates to a plasmid vector that, when transiently or permanently transfected into mammalian cell lines, enables the synthesis of the relevant protein by these mammalian cells using tailored gene information. The plasmid vector according to the invention is suitable for use in scientific research for the production of proteins in the fields of pharmaceuticals and biotechnology.

### BACKGROUND

Plasmid vectors, which enable the expression of protein-coding genes in mammalian cells, have a history of more than 30 years. The concept of producing protein in mammalian cells using a plasmid vector and the properties that the plasmids used for this purpose are known in the art. The basic properties of such plasmids fall within the scope of general knowledge. There are numerous papers and patents in the scientific literature on such vectors. Several mammalian cell expression plasmids are commercially available. These plasmids can produce proteins in mammalian cells for different purposes. In the state of the art, the ability of freely available plasmids to produce proteins in mammalian cells, i.e., the amount of protein that can be produced per cell under appropriate culture conditions and over a certain time, is not at the desired level.

Patent No. EP0332416A2 relates to a plasmid containing pCMV LacI DNA sequence and is used for protein expression in mammalian cells. The related patent protects the conditions that enable the expression of the relevant protein to be regulated in mammalian cells, that is, the expression or absence of expression according to desire.

Patent No. EP2227546B 1 describes methods for expressing a relevant polypeptide in a mammalian host cell using a mammalian expression vector to express a relevant polypeptide, as well as host cells containing another relevant vector and said vector.

According to the invention in Patent No. WO2019157239A1 a mammalian expression plasmid vector includes (a) a prokaryotic source of replication; (b) a suitable eukaryotic promoter for expression of one or more transgenes; (c) a multiple cloning site for insertion of one or more transgenes; and (d) a eukaryotic promoter and a prokaryotic promoter, includes a nucleic acid encoding a selectable marker operably linked to a binary promoter. It contains dihydrofolate reductase (DHFR), multiple cloning sites (MCS), and puromycin resistance gene. The property of said plasmid and related methods put forward is the integration of large nucleotide sequences (more than 5 kb) into the target cell DNA.

### SUMMARY

The primary object of the present invention is to achieve high production of the relevant protein in the relevant cells.

The object of the present invention is to develop a gene expression plasmid that enables protein production in mammalian cells for use in scientific research and industrial activities open to commercial applications.

The plasmid according to the invention contains Cytomegalovirus (Cytomegalovirus, CMV) promoter and enhancer, CMV intron A, small t intron and simian vacuolating virus 40 (SV40) polyA sequences from the pCMV LacI vector that has been described in the 90s, as shown in FIG. 2. Additions were made to the DNA sequences described above to increase the production of the respective proteins. These additions are a synthetic AT-rich 21-bp sequence (Syn21) and human telomerase reverse transcriptase (hTERT)-SV40 enhancers and have been published in different articles before (Pfeiffer et al., 2012; Watanabe et al., 2014). However, with the invention, for the first time, these additions were combined with pCMV LacI elements, thus providing high production of a high amount of the protein of interest. The plasmid of the invention also contains the puromycin antibiotic resistance gene for selection in mammalian cells and the DHFR gene for amplification in DHFR negative cells. In conclusion, the plasmid of the invention contains CMV promoter and enhancer, CMV intron A, small t intron and SV40 polyA, Syn21 and hTERT-SV40 enhancer, puromycin resistance and DHFR gene elements.

Plasmid pDAD2.0, the core of the invention, produces higher amounts of protein compared to some commercially available plasmids under similar conditions. Therefore, it is a suitable plasmid for protein production under high-yield conditions, especially in the pharmaceutical industry. The reason for the higher production of the related protein is the addition of Syn21 and hTERT-SV40 enhancer elements in addition to CMV promoter and enhancer, CMV intron A, small t intron, and SV40 polyA elements. Western blot (top side) and ELISA (bottom side) methods shown in FIGS. 3A-3C demonstrates that the plasmid pDAD2.0 yielded a higher protein production than an existing commercial plasmid (pcDNA3.1, Invitrogen). The elements used to produce the relevant protein in the pcDNA3.1 plasmid used in the comparative analyses are CMV enhancer, CMV promoter, and bGH polyA signal sequences, respectively. On the other hand, the elements used to produce the relevant protein in the plasmid of the invention are CMV enhancer, CMV promoter, CMV intron A, Syn21, small t intron, SV40 polA signal, and hTERT-SV40 enhancer, respectively. When both plasmids are compared, CMV enhancer and CMV promoter sequences show close to one hundred percent similarity, but poly A signals in both plasmids are not similar. In addition, the pDAD2.0 plasmid contains a sequence of CMV intron A, Syn21, small t intron, and hTERT-SV40 enhancer units, which is not found in pcDNA3.1 plasmid and has a total nucleotide length of 1419.

The principal DNA sequence information of the vector of the invention consists of the plasmid pCMV LacI (GenBank: U64448.1; submitted 1996) from the Lacswitch II system. Nucleotides 2690-3801 and 4794-5824 were deleted from this DNA sequence consisting of 7995 nucleotides. In order to increase the protein expression power of the vector, DNA sequences were added to the regions indicated in Table 1 as the Syn21 (Pfeiffer et al., 2012) and hTERT-SV40 enhancer (Watanabe et al., 2014) regions. The Puromycin resistance gene was added to this vector under the control of the HSV TK promoter in order to generate mammalian cells that can continuously produce the relevant proteins. In addition, the DNA sequence encoding DHFR was added under the control of the PGK promoter for production in mammalian cells such as CHO DG44, which is deficient in the DHFR gene. The plasmid also contains the Ampicillin resistance gene and bacterial Ori DNA sequences from the pUC57 plasmid for replication and cloning in bacterial cells. A multiple cloning site was also added to the plasmid to allow the desired genes to be included in the plasmid. This plasmid has been tested in mammalian cells for the capacity to produce different proteins temporarily or permanently, and successful results have been obtained. In FIG. 3A, the production of human immunoglobulin G1 (IgG1) constant region (Fc) protein in fusion with the SARS-COV2 virus receptor binding region (RBD) (Protein ID: PODTC2; aa 333-529) was tested. In FIGS. 3B-3C, the production capacity of the fusion human IgG1 Fc (Protein ID: P01857; aa 99-330), i.e., ACE2-Fc protein, with the human ACE2 protein extracellular region (Protein ID: Q9BYF1; aa 1-740) was tested.

The plasmid pDAD2.0 of the invention has the potential to be used in scientific research and industrial production to express desired proteins in mammalian cells. This plasmid can be used for the production of any protein that has commercial value and can be synthesized in mammalian cells. In particular, many recombinant protein-based drugs are still industrially produced in CHO cells lacking the DHFR gene. The pDAD2.0 plasmid is an ideal plasmid for such production.

### BRIEF DESCRIPTION OF THE DRAWINGS

FIG. 1: shows the complete nucleotide sequence of plasmid pDAD2.0 (SEQ ID NO: 1). It is given linearly in fasta format.

FIG. 2: shows the map of the pDAD2.0 plasmid. Drawn from the DNA sequence using SnapGene® software. a. Elements that are essential for target gene transcription and translation and play an enhancer role. While the promoter and poly(A) are essential regions, the enhancer and intronic regions play an enhancer role. b. Puromycin resistance (PuroR) and the DHFR gene cassette play a role in the selection of mammalian 1 cells. c. The starting point for prokaryotic replication is Ori, and the selection gene cassette ampicillin (AmpR), which provides amplification of the plasmid.

FIGS. 3A-3C: Comparison of protein expression provided by plasmids pDAD1.0 and pDAD2.0 with that of commercial plasmids. Two different structures of protein cloned into pDAD1.0, pDAD2.0 plasmid, and commercial pcDNA3.1 plasmid (Thermo Invitrogen) containing the same DNA sequence were compared. Among the examples used here, ACE2 is a human protein, while RBD is a virus protein. FIG. 3A) Human immunoglobulin G1 (IgG1) constant region (Fc) (Protein ID: P01857; aa 104-330) recombinant protein RBD-Fc in fusion with the SARS-COV2 virus receptor binding domain (RBD) (Protein ID: PODTC2; aa 333-529) FIGS. 3B-3C) comparison of the capacity of human IgG1 Fc (Protein ID: P01857; aa 99-330) fused with human ACE2 protein extracellular domain (Protein ID: Q9BYF1; aa 1-740) to produce ACE2-Fc recombinant protein by Western blot and ELISA techniques.

FIG. 3A (Upper side) Western blot result against human IgG in reducing conditions with protein lysate and supernatant samples obtained 72 hours after HEK293 cells expressing the RBD-Fc fusion ectopically. 30 μl of the sample was loaded into the wells at the collection including the 4× protein loading dye. FIG. 3B (upper side). Western blot results against human IgG in reducing conditions with protein lysate and supernatant samples obtained 72 hours after HEK293 cells expressing the ACE2-Fc fusion ectopically. 32 μl of the sample was loaded into the wells at the collection including the 4× protein loading dye. FIG. 3C (upper side). Western blot results against human IgG in reducing conditions with protein lysate and supernatant samples obtained 72 hours after HEK293T cells expressing the ACE2-Fc fusion ectopically. 32 μl of the sample was loaded into the wells at the collection including the 4× protein loading dye. FIGS. 3A-3C (Bottom side) ELISA plates were coated using 100 μl per well of 4 μg/ml anti-human IgG goat antibody solution (AffiniPure, Jackson ImmunoResearch). Cell supernatant samples were applied to the wells at different dilution ratios of 1:10, 1:100, and 1:1000. Colorimetric signal was measured at 450 nm after incubation with peroxidase conjugated (HRP) anti-human IgG1 antibodies.

### DETAILED DESCRIPTION OF THE EMBODIMENTS

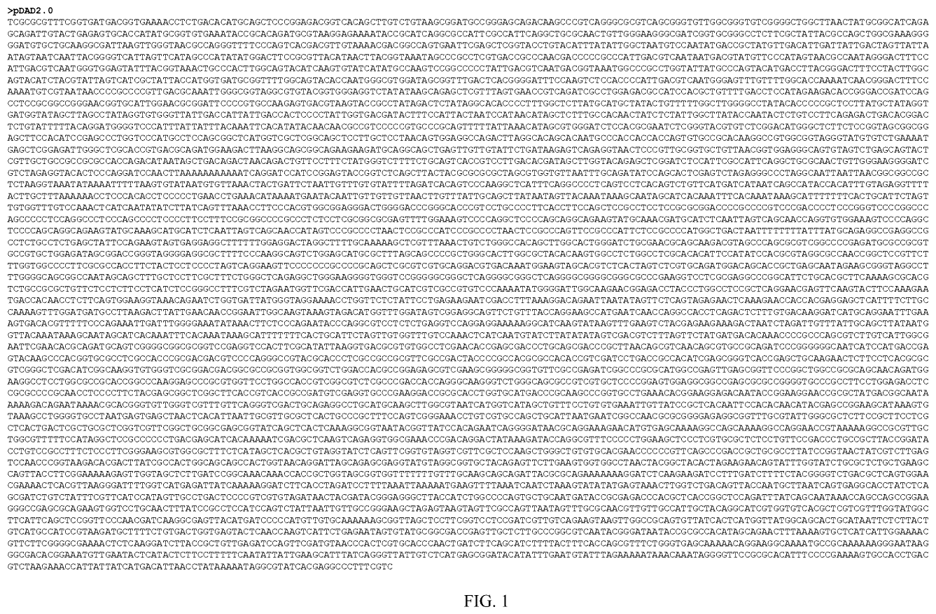

The Plasmid vector of the invention contains the nucleic acid sequence that is at least 80% identical to the SEQ ID NO: 1 shown in FIG. 1.

pDAD2.0 is designed as a vector consisting of 7587 nucleotides that can be replicated in bacterial cells and used for protein production in mammalian cells. The entire nucleotide sequence is clearly presented in FIG. 1. In order to obtain this structure, pCMV LacI plasmid DNA information from the Lacswitch II system (GenBank: U64448.1; submitted 1996) was the main source. Nucleotides 1-997 and 2690-3801 and nucleotides 4794-5824 and 5915-7995 were deleted from the pCMV LacI DNA sequence consisting of 7995 nucleotides. In order to obtain the pDAD2.0 plasmid, various DNA sequences were appropriately added to the remaining DNA sequences, while many nucleotides of the DNA from pCMV LacI were changed. The general purpose of these changes is to increase the protein production of the plasmid in mammalian cells to a high capacity and to keep the plasmid in mammalian cells continuously when necessary. For this purpose, the following actions have been taken.

The plasmid vector includes;
Cytomegalovirus (CMV) promoter containing the sequence specified at nucleotides 836 to 1040 in SEQ ID NO: 1,
Cytomegalovirus (CMV) enhancer containing the sequence specified at nucleotides 456 to 835 in SEQ ID NO: 1,
CMV intron A, containing the sequence specified at nucleotides 1176 to 1997 in SEQ ID NO: 1,
SV40 small t antigen intron containing the sequence specified at nucleotides 2247 to 2312 in SEQ ID NO: 1,
SV40 poly(A) containing the sequence specified at nucleotides 2471 to 2592 in SEQ ID NO: 1,
Syn21 containing the sequence specified at nucleotides 2106 to 2130 in SEQ ID NO: 1,
hTERT-SV40 enhancer containing the sequence specified at nucleotides 2604 to 3111 in SEQ ID NO: 1.

The plasmid vector according to the invention also includes the Ampicillin resistance gene containing the sequence specified at nucleotides 6527 to 7387 in SEQ ID NO: 1 or kanamycin, spectinomycin, streptomycin, carbenicillin, bleomycin, puromycin, erythromycin, polymyxin B, tetracycline, hygromycin chloramphenicol or an equivalent resistance gene for plasmid selection in prokaryotic cells for use in its place. The plasmid vector according to the invention also includes the Puromycin resistance gene containing the sequence specified at nucleotides 4633 to 5232 in SEQ ID NO: 1, or blasticidin, G418/geneticin, hygromycin B, zeocin, neomycin, or an equivalent resistance gene, which enables the selection of cells containing the plasmid in mammalian cells for use in its place.

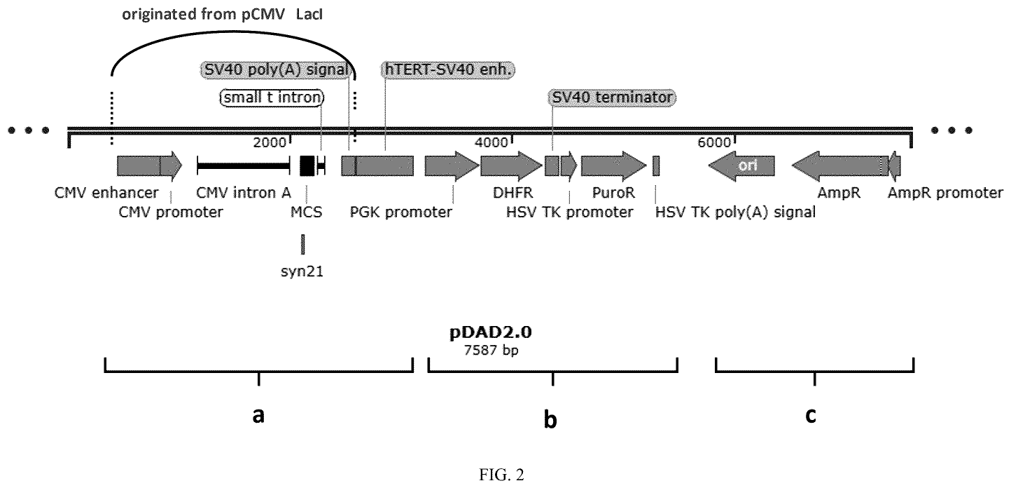

The present invention is a plasmid vector characterized by including the dihydrofolate reductase (DHFR) gene, which contains the sequence specified at nucleotides 3729 to 4292 in SEQ ID NO: 1, or the glutamine synthetase (GS) gene or another equivalent gene, which provides cell survival depending on the host cell for use in its place. The purpose of use of the structures in the pDAD2.0 plasmid can be summarized in 3 main categories. There are transcription cassettes required for the production of the RNA sequence (mRNA) of the target gene, and sequences that increase the number of RNA copies from the target gene, and also help to express more protein by increasing the translation power from RNA to protein under the group as shown in FIG. 2. CMV enhancer, CMV promoter, CMV intron A, Syn21, small t intron, SV40 poly(A) signal, and hTERT-SV40 enhancer are the elements belonging to this group. There is also a multiple cloning site in this region to clone the target gene. Necessary elements (puromycin resistance gene and DHFR) to keep the plasmid in the mammalian cell when necessary and to use the same in protein expression were collected in category b. PGK promoter, DHFR gene, SV40 terminator, HSV TK promoter, puromycin resistance gene (PuroR), and HSV TK polyA signal elements are included in this category. In order to reproduce the plasmid in the prokaryotic system and to select plasmid-producing bacteria, the ampicillin resistance gene, and the Ori element, which provides prokaryotic replication, are also in the c category. This group includes the Ori element, along with the ampicillin promoter and resistance gene. In the development stages of the resulting product, Syn21 (Pfeiffer et al., 2012), hTERT-SV40 enhancer (Watanabe et al., 2014), WHBV (Woodchuck hepatitis B virus) post-transcriptional regulatory element (Zufferey et al., 1999; GenBank: J04514.1) and p10-UTR (Smith et al., 1983) DNA sequences were added to the regions mentioned in Table 1. In addition, the DNA sequence encoding the Puromycin resistance gene under the control of the HSV TK promoter and the DHFR gene (dihydrofolate reductase enzyme) under the control of the PGK promoter were added to the nucleotide regions indicated in Table 1. The plasmid also contains the Ampicillin resistance gene and bacterial Ori DNA sequences from plasmid pUC57 (GenBank: Y14837.1). The plasmid designed in this way also includes appropriate restriction enzyme cleavage sites and nucleotide modifications to silence off-target translations. After this sequence was designed, it was commercially synthesized by GenScript and plasmid pDAD1.0 was obtained. This early version (precursor) of our plasmid was compared with commercial plasmids and better results were obtained in terms of protein expression. Subsequently, modifications that increase protein expression were performed on pDAD1.0, the plasmid pDAD2.0 with the best production under current conditions is obtained. These modifications firstly included deletion of the WHBV post-transcriptional regulatory element by cutting with PacI restriction enzyme and re-ligation. This intermediate plasmid was tested for ACE2-Fc production, and it has been observed that it provides more ACE2-Fc protein production than pDAD1.0. In addition, it was observed that ACE2-Fc production was higher with the pDAD plasmid obtained after the p10-UTR region was removed from pDAD1.0 by cutting with the NotI enzyme compared to pDAD1.0. For this reason, the pDAD form without both WHBV and p10-UTR regions was created and named pDAD2.0. FIG. 2 shows the final fragments that make up the pDAD2.0 plasmid. Table 1 summarizes the nucleotide locations where the structures are found with reference to the sequence given in FIG. 1.

TABLE 1
pDAD2.0 plasmid elements and their respective locations

| Plasmid Element | Plasmid Nucleotide Location |
|---|---|
| CMV enhancer | 456-835 |
| CMV promoter | 836-1040 |
| CMV intron A | 1176-1997 |
| Multiple cloning site | 2132-2240 |
| Syn21 | 2106-2130 |
| SV40 small t antigen intron | 2247-2312 |
| SV40 poly(A) signal | 2471-2592 |
| hTERT-SV40 enhancer | 2604-3111 |
| PGK promoter | 3223-3722 |
| DHFR gene cDNA | 3729-4292 |
| SV40 terminator | 4299-4418 |
| HSV TK promoter | 4445-4590 |
| Puromycin resistance cDNA | 4633-5232 |
| HSV TK poly(A) signal | 5273-5321 |
| Replication origin | 5768-6356 |
| Ampicillin resistance cDNA | 6527-7387 |
| AmpR promoter | 7388-7492 |

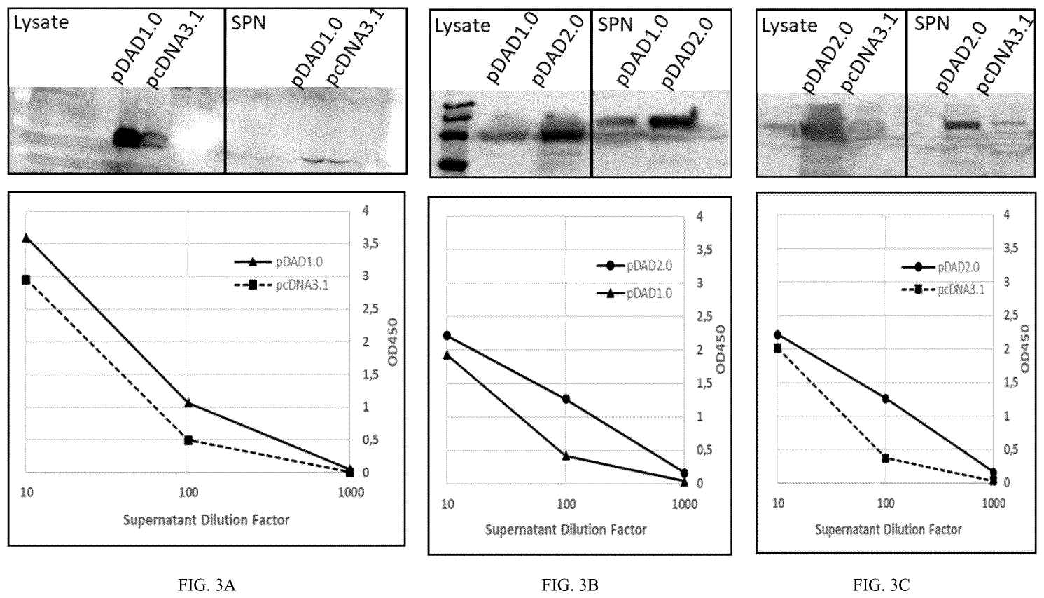

The plasmid pcDNA3-SARS-COV-2-S-RBD-Fc (Addgene #141183, Chan et al., 2020) derived from the commercially available plasmid pcDNA3.1 was used as a comparative to demonstrate the capacity of the construct, called the precursor plasmid pDAD1.0, to produce the targeted protein. Sequences containing the SARS-COV2 virus receptor binding region (RBD) (Protein ID: PODTC2; aa 333-529) and the human immunoglobulin G1 (IgG1) Fc region (Protein ID: P01857; aa 104-330) in this plasmid were transferred to plasmid pDAD1.0 by restriction enzymes. Western blot and ELISA experiments were performed to compare the efficacy of pDAD1.0 vs pcDNA3.1 with cell lysate and supernatant samples collected 72 hours after ectopic administration to HEK293 cells. As shown at the top in FIG. 3A, pDAD1.0 performed better than pcDNA3.1 for the production of RBD-Fc protein both intracellularly and extracellularly. These results were quantitatively confirmed by the ELISA test (FIG. 3A bottom). For comparison of pDAD2.0 with pDAD1.0, the pcDNA3.1_ACE2-Fc plasmid derived from the pcDNA3.1 plasmid containing the fusion human IgG1 Fc (Protein ID: P01857; aa 99-330) structure with the human ACE2 protein extracellular region (Protein ID: Q9BYF1; aa 1-740) was used. As shown in FIG. 3B pDAD2.0 performed better than pDAD1.0 for the expression of intracellular and secreted ACE2-Fc. Finally, it was tested in HEK293T cells, where the obtained pDAD2.0 plasmid had better production capacity than the still existing pcDNA3.1 plasmid, and the results are presented in FIG. 3C in comparison with the original ACE2-Fc plasmid.

What is claimed is:

1. A plasmid vector, comprising a nucleic acid sequence with at least 80% similarity to SEQ ID NO: 1.

2. The plasmid vector according to claim 1, comprising:
a cytomegalovirus (CMV) promoter containing the sequence specified at nucleotides 836 to 1040 in SEQ ID NO: 1,
a CMV enhancer containing the sequence specified at nucleotides 456 to 835 in SEQ ID NO: 1,
CMV intron A containing the sequence specified at nucleotides 1176 to 1997 in SEQ ID NO: 1,
simian vacuolating virus 40 (SV40) small t antigen intron containing the sequence specified at nucleotides 2247 to 2312 in SEQ ID NO: 1,
an SV40 poly(A) signal containing the sequence specified at nucleotides 2471 to 2592 in SEQ ID NO: 1,
a synthetic AT-rich 21-bp sequence (Syn21) containing the sequence specified at nucleotides 2106 to 2130 in SEQ ID NO: 1,
a human telomerase reverse transcriptase (hTERT)-SV40 enhancer containing the sequence specified at nucleotides 2604 to 3111 in SEQ ID NO: 1.

3. The plasmid vector according to claim 2, further comprising an ampicillin resistance gene containing the sequence specified at nucleotides 6527 to 7387 in SEQ ID NO: 1, or kanamycin, spectinomycin, streptomycin, carbenicillin, bleomycin, puromycin, erythromycin, polymyxin B, tetracycline, hygromycin chloramphenicol, or an equivalent resistance gene playing a same role as the ampicillin resistance gene in a plasmid selection in prokaryotic cells.

4. The plasmid vector according to claim 2, further comprising a puromycin resistance gene containing the sequence specified at nucleotides 4633 to 5232 in SEQ ID NO: 1, or blasticidin, G418/geneticin, hygromycin B, zeocin, neomycin, or an equivalent gene playing a same role as the puromycin resistance gene in selecting cells containing the plasmid vector in mammalian cells.

5. The plasmid vector according to claim 2, further comprising a dihydrofolate reductase (DHFR) gene containing the sequence specified at nucleotides 3729 to 4292 in SEQ ID NO: 1, or a glutamine synthetase (GS) gene, or an equivalent gene playing a same role as the DHFR gene in cell survival depending on a host cell.

6. The plasmid vector according to claim 2, further comprising a multiple cloning site (MCS) at nucleotides 2132 to 2240 in SEQ ID NO: 1, wherein the MCS contains a plurality of restriction enzyme sites, the plurality of restriction enzyme sites allow an addition of a gene of interest into the plasmid vector.
